(19) United States
(12) Patent Application Publication
(10) Pub. No.: US 2024/0327439 A1
(43) Pub. Date: Oct. 3, 2024

(54) PHARMACOLOGICAL AGENTS FOR PREVENTING AND TREATING CATARACTS AND PRESBYOPIA EYE DISEASES

(71) Applicant: Plex Pharmaceuticals, Inc., San Diego, CA (US)

(72) Inventors: Santosh C. SINHA, Ladera Ranch, CA (US); Sridhar Govinda PRASAD, San Diego, CA (US)

(21) Appl. No.: 18/575,218

(22) PCT Filed: Jun. 28, 2022
(86) PCT No.: PCT/US2022/035336

§ 371 Date: Dec. 28, 2023

Related U.S. Application Data
(60) Provisional application No. 63/215,818, filed on Jun. 28, 2021.

(51) Int. Cl. C07F 9/38; A61K 31/222; A61K 31/661; A61K 31/662; C07C 205/45; C07F 9/12

(52) U.S. Cl. CPC C07F 9/38; A61K 31/222; A61K 31/661; A61K 31/662; C07C 205/45; C07F 9/12

(57) ABSTRACT

Methods of treating presbyopia or cataract in a subject in need thereof are provided. The methods require administering to the subject an effective amount of a composition comprising a compound that inhibits the formation or dissolves high molecular weight aggregates of human α-A-crystallin. Compositions containing certain compounds are believed to be also effective in the treatment of transthyretin (TTR)-associated amyloidosis, Prion, Creutzfeldt-Jakob, Gerstmann-Sträussler-Scheinker disease and Ankyloblepharon-ectodermal dysplasia-cleft lip/palate syndrome.

### CROSS-REFERENCE TO RELATED APPLICATIONS

This application claims the benefit of U.S. Provisional Application Ser. No. 63/215,818, which was filed on Jun. 28, 2021, and the entire contents of which is expressly incorporated by reference herein.

### FIELD OF THE DISCLOSURE

Disclosed herein are 4-Phosphono-butyric acid 2-hydroxy-5-(4-methyl-benzoyl)-3-nitro-phenyl ester sodium salt and other compounds useful to treat presbyopia or cataract eye diseases.

### BACKGROUND

Cataracts are the leading cause of blindness (51%) worldwide according to the World Health Organization (WHO), particularly in low- and middle-income countries. Data dating back to the beginning of this millennium showed that 30-60% of blindness in Africa and 60-80% in South East Asia is attributable to cataracts. In the United States, the current number of those with cataract is estimated to be more than 25.7 million. Projections from Prevent Blindness research estimate that the number will increase to 38.5 million by 2032, and to 45.6 million by the year 2050. Cataract is a clouding of the eye's lens which blocks or changes the passage of light into the eye. Cataracts usually form in both eyes, but not at the same rate. They can develop slowly or quickly, or progress to a certain point, then not get any worse. Besides aging, other factors may cause cataracts to form. Eye infections, some medicines (such as steroids), smoking, injuries, trauma, or exposure to intense heat or radiation may cause cataracts. Too much exposure to non-visible sunlight (called UV or ultraviolet light) and various diseases, such as diabetes or metabolic disorders, may also contribute to cataracts formation.

The only treatment currently available is surgical extraction of the lens and replacement with an interocular lens that is accompanied by a high public health burden. Although cataract surgery is generally considered to be safe, there are significant complications: (i) 30-50% of patients in the US having cataract surgery develop opacification of the posterior lens capsule within two years and require laser treatment; (ii) 0.8% have retinal detachments; (iii) 0.6-1.3% are hospitalized for corneal edema or require corneal transplantation and (iv) about 1% are presented with endophthalmitis. In addition, in many remote and poor areas of the developing and under-developed regions of the world, people still remain blind from cataract, primarily due to lack of access to eye care.

Presbyopia is the loss of accommodative ability of the eye resulting in the inability to focus on near objects. Presbyopia affects everyone over the age of 45 and has significant negative impacts on the quality of life. Current treatments for presbyopia include: (i) non-invasive approaches that utilize devices to help improve near and distance vision but do nothing to restore the natural process of accommodation and require constant use of the devices, and (ii) invasive surgical procedures which are associated with major complications including decrease in vision quality, regression effects, anisometropia, corneal ectasia, and haze. Most importantly, none of these methods can reverse presbyopia. Moreover, no treatment option exists that can either prevent or delay the onset of presbyopia.

Stiffening of eye lens and changes in the elasticity of the lens capsule, dimension of eye lens, dimension of the zonular attachment, and ciliary muscle (CM) contractions, have all been proposed as contributing factors for presbyopia. However, human and non-human primate studies suggest that CM function is normal well beyond the onset of presbyopia. By contrast, the human lens increases in stiffness with age in a manner that directly correlates with a loss in accommodative power. The loss in accommodative power can be restored by implanting intraocular lenses made from a flexible polymer suggesting that restoration of lens flexibility is sufficient to restore accommodation. Therefore, a pharmacological agent that could prevent or reverse the hardening of the crystalline lens would provide a promising avenue for a novel non-invasive treatment for presbyopia.

At the molecular level, proteins known as crystallins play a major role in the stiffening of the eye lens. The lens crystallins comprise three isoforms, α, β, and γ and make up 90% of the eye lens protein content. α crystalline (AC), an ATP-independent chaperone and member of the small heat shock protein (sHsp) family, constitutes 40% of the crystallin protein content. It exists as a hetero-oligomer of two subunits, αA-crystallin (AAC) and αB-crystallin (ABC) and its expression is primarily restricted to the eye lens. It recognizes exposed conformational features in partially unfolded lens proteins and sequesters them from one another, thereby reducing the population of aggregation-prone species that would otherwise lead to various age-related vision impairment.

Multiple studies have established a link between stiffening of the human lens and AC function. Dynamic mechanical analysis measurements have shown that there is a significant increase in the stiffness of the lens with age, particularly in the lens nucleus where a 500- to 1000-fold decrease in elasticity is observed. This increase in lens stiffness correlates with the age-related decline in free AC chaperone concentration as most AC becomes incorporated into high molecular weight (HMW) aggregates by the age of 40-50. This conversion of soluble AC into HMW aggregates is accompanied by a large increase in lens stiffness, presumably because the low level of soluble AC present is not sufficient to chaperone denatured proteins. That age-related decrease in free AC chaperone is responsible for lens stiffness is supported by experiments where human lenses were subjected to heating to mimic the age-related conversion of soluble AC into HMW aggregates and an increase in lens stiffness was observed. Similarly, purified soluble AC forms HMW aggregates when exposed to UV radiation with a loss in chaperone like activity. The HMW aggregate is formed due to the intermolecular cross-linking, particularly S—S bonds, resulting from the oxidation of cysteine sulfhydryl groups (—SH). The formation of this disulfide cross-linked HMW aggregate is thought to be a major contributor in increasing the stiffness and loss of accommodation amplitude of the lens.

It has been suggested that presbyopia is the earliest observable symptom of age-related nuclear (ARN) cataract, a major cause of blindness in the world.

Given the need for noninvasive treatment that can protect and restore the accommodative ability of the eye lost in presbyopia and given that formation of HMW AC aggregates is a major causative factor underlying presbyopia, there is a need for the development of pharmacological agents that can selectively delay and/or reverse the HMW AC aggregate formation.

### DESCRIPTION

Provided herein is a rational structure activity relation-based approach for identifying small molecule disaggregases (SMDs) that can inhibit the formation and/or dissolve HMW aggregates of human ACC (hAAC). Several SMDs were identified based on this approach. It is believed that these SMDs are useful for the treatment and management of presbyopia, and for the treatment and/or slowing down the progression of cataract. The cataract can be age-related (nuclear sclerotic, cortical, and posterior subcapsular), congenital, familial, secondary, traumatic, smoke-related and radiation cataracts.

In one aspect, provided herein is a compound for treating or managing presbyopia or slowing down the progression and/or treating cataract in a subject in need thereof. The compound comprises administering to the subject an effective amount of a composition comprising a compound having the formula (I)

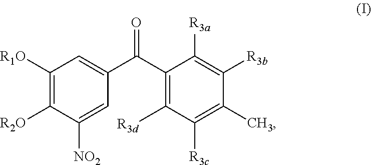

or a solvate or a pharmaceutically acceptable salt thereof,
wherein,
R₁ and R₂ are the same or different, and each of R₁ and R₂ is independently selected from the group consisting of: hydrogen, R₄C═O,

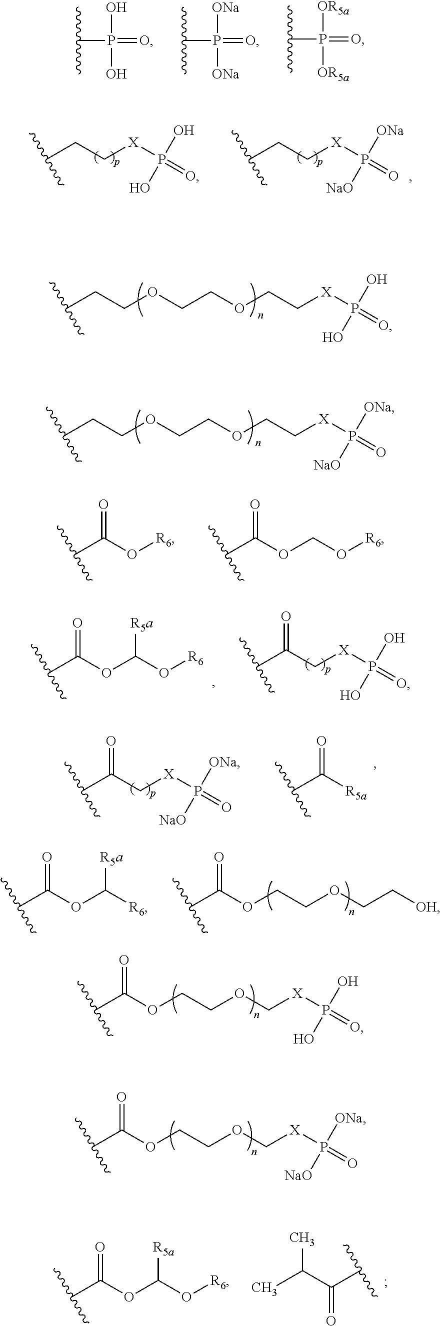

R₃ₐ, R_{3b}, R_{3c}, and R_{3d} are each the same or different, and each independently are selected from the group consisting of: hydrogen, branched or linear (C₁-C₆)alkyl, halo(C₁-C₆)alkyl, (C₃-C₆)cycloalkyl, halo(C₃-C₆)cycloalkyl, and hydroxyl;
R₄ is selected from the group consisting of: branched or linear (C₁-C₆)alkyl; halo(C₁-C₆)alkyl; (C₃-C₆)cycloalkyl; halo(C₃-C₆)cycloalkyl; aryl; haloaryl; and
R₅ₐ and R_{5b} are each the same or different, and independently a branched or linear (C₁-C₆)alkyl,
R₆ is branched or linear (C₁-C₆)alkyl, aryl, or a polyethylene glycol group or

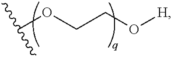

wherein q is 1 to 10;
p is a number from 0 to 10,
n is a number from 0 to 10, and
X is a C or O.

In some aspects, R₁ and R₂ are the same. In some aspects, at least one of R₁ and R₂ is hydrogen. In some aspects, at least one of R₃ₐ, R_{3b}, R_{3c}, and R_{3d} is hydrogen. In some aspects, In some aspects, each of R₃ₐ, R_{3b}, R_{3c}, and R_{3d} is hydrogen.

In one aspect, provided is a compound of Formula (1), wherein at least one of R₁ and R₂ is selected from the group consisting of

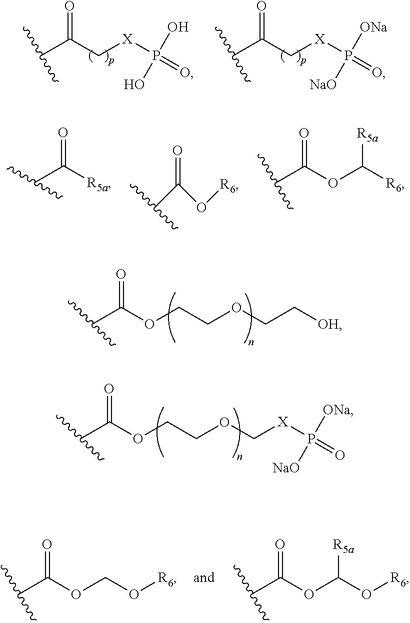

wherein R₅ₐ is (C₁-C₆)alkyl and R₆ is (C₁-C₆)alkyl, aryl, or,

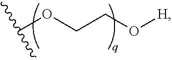

wherein q is 1 to 10,
or a solvate or a pharmaceutically acceptable salt thereof.

In one aspect, provided is a compound of formula (I), wherein:
one of R₁ and R₂ is

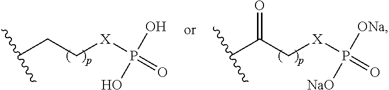

wherein X is C and p is 1 or 2,
the other of R₁ and R₂ is hydrogen, and
each of R₃ₐ, R_{3b}, R_{3c}, and R_{3d} is hydrogen.

In one aspect, provided is a compound of formula (I), wherein:
one of R₁ and R₂ is R₄C═O, wherein R₄ is a branched (C₃-C₆)alkyl, such as isopropyl,
the other of R₁ and R₂ is hydrogen; and
each of R₃ₐ, R_{3b}, R_{3c}, and R_{3d} is hydrogen. In one aspect, R₁ is R₄C═O, wherein R₄ is isopropyl and each of R₂, R₃ₐ, R_{3b}, R_{3c}, and R_{3d} is hydrogen.

In one aspect, provided is a compound of formula (I), wherein:
R₁ and R₂ are the same or different, and are each independently R₄C═O, wherein R₄ is a branched (C₃-C₆)alkyl, such as isopropyl,
each of R₃ₐ, R_{3b}, R_{3c}, and R_{3d} is hydrogen. In one aspect, R₁ and R₂ are each R₄C═O, wherein R₄ is isopropyl, and each of R₃ₐ, R_{3b}, R_{3c}, and R_{3d} is hydrogen.

In one aspect, provided is a compound of formula (I), wherein:
R₁ and R₂ are the same or different, and are each independently

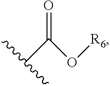

wherein R₆ is a branched (C₃-C₆)alkyl, such as isopropyl,
each of R₃ₐ, R_{3b}, R_{3c}, and R_{3d} is hydrogen.

In one aspect, provided is a compound of formula (I), wherein:
one of R₁ and R₂ is

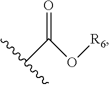

wherein R₆ is a branched (C₃-C₆)alkyl, such as isopropyl,
the other of R₁ and R₂ is hydrogen;
each of R₃ₐ, R_{3b}, R_{3c}, and R_{3d} is hydrogen. In some aspects, R₁ is

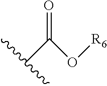

wherein R₆ is isopropyl, and each of R₂, R₃ₐ, R_{3b}, R_{3c}, and R_{3d} is hydrogen.

In another aspect, provided herein is the following compound of formula (Ia):

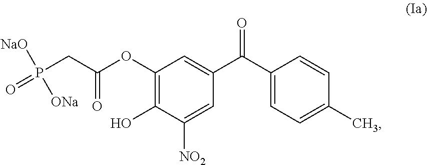

or a solvate or a pharmaceutically acceptable salt thereof.

In another aspect, provided herein is a compound of formula (Ib):

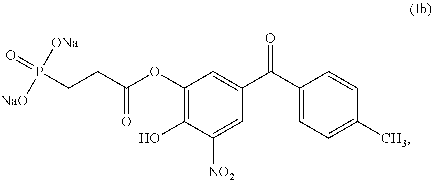

or a solvate or a pharmaceutically acceptable salt thereof.

In another aspect, provided herein is a compound of formula (Ic):

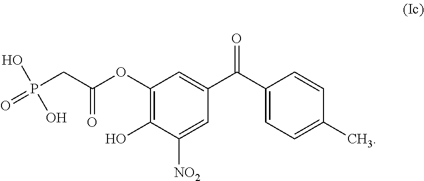 [SMILES: Cc1ccc(C(=O)c2cc(OC(=O)CP(=O)(O)O)c(O)c([N+](=O)[O-])c2)cc1]

or a solvate or a pharmaceutically acceptable salt thereof.

In another aspect, provided herein is a compound of formula (Id):

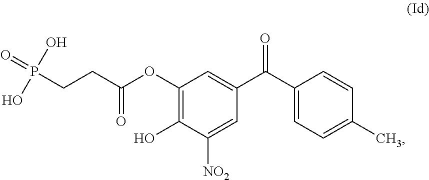 [SMILES: Cc1ccc(C(=O)c2cc(OC(=O)CCP(=O)(O)O)c(O)c([N+](=O)[O-])c2)cc1]

or a solvate or a pharmaceutically acceptable salt thereof.

In another aspect, provided herein is a compound of formula (Ie):

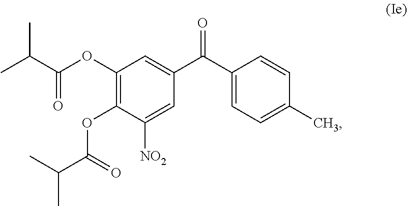 [SMILES: Cc1ccc(C(=O)c2cc(OC(=O)C(C)C)c(OC(=O)C(C)C)c([N+](=O)[O-])c2)cc1]

or a solvate or a pharmaceutically acceptable salt thereof.

In another aspect, provided herein is a compound of formula (If):

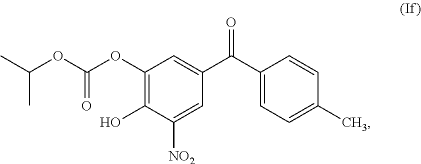 [SMILES: Cc1ccc(C(=O)c2cc(OC(=O)OC(C)C)c(O)c([N+](=O)[O-])c2)cc1]

or a solvate or a pharmaceutically acceptable salt thereof.

In another aspect, provided are the following compounds:

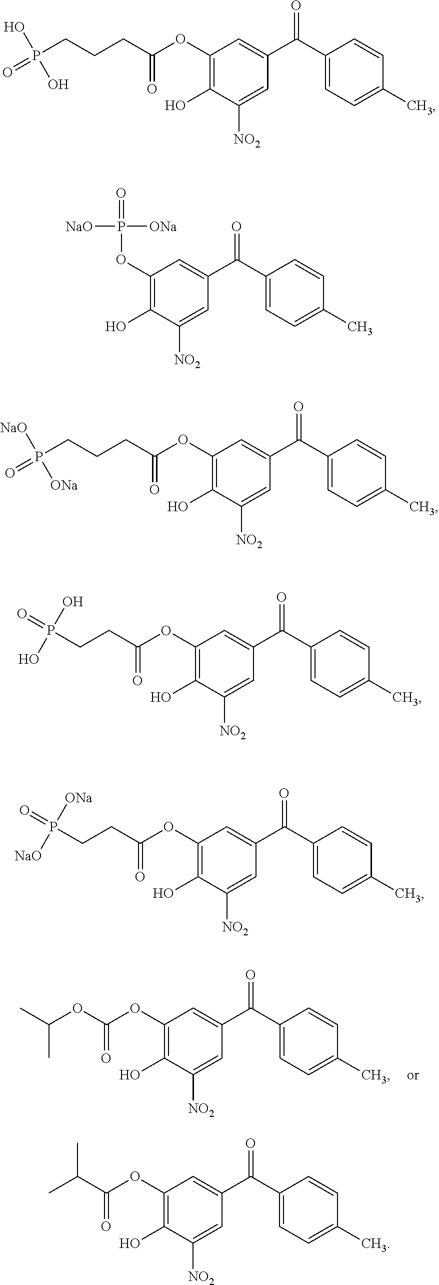

or a solvate or a pharmaceutically acceptable salt thereof.

In one aspect, the compounds of formula (I) are produced by the following general reaction:

### General Scheme for Making Prodrugs:

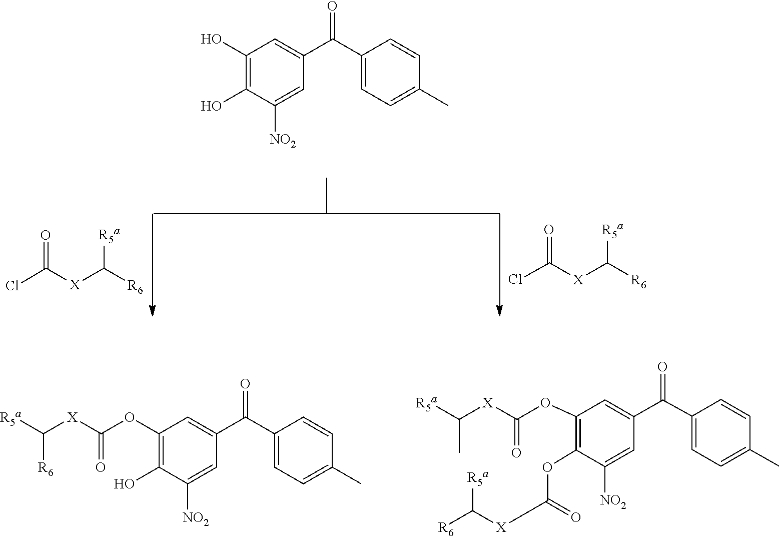

In one aspect, provided is a method of treating, preventing, reducing the occurrence of, or reducing, ameliorating, or alleviating the symptoms associated with presbyopia, cataract, transthyretin (TTR)-associated amyloidosis, or other conditions or disorders associated with the eye, comprising administering to a subject in need thereof, an effective amount of the compound of formula (I), including each of the disclosed compounds and the compounds of formula (Ia), (Ib), (Ic), (Id), (Ie), and (If). In some aspects, provided is a pharmaceutical composition comprising a compound of formula (I), including each of the disclosed compounds and the compounds of formula (Ia), (Ib), (Ic), (Id), (Ie), and (If), and one or more pharmaceutically excipients. In some aspects, the compound of formula (I), including each of the disclosed compounds and the compounds of formula (Ia), (Ib), (Ic), (Id), (Ie), and (If), may be administered to a subject in need thereof, in an amount effective to reduce or inhibit the formation of, or dissolve high molecular weight aggregates of human α-A-crystallin, or to treat, prevent, or reduce the occurrence of or to reduce, ameliorate, or alleviated symptoms associated with conditions relating to human α-A-crystallin. In some aspects, the conditions include but are not limited to: transthyretin (TTR)-associated amyloidosis, Prion, Creutzfeldt-Jakob, Gerstmann-Sträussler-Scheinker disease and Ankyloblepharon-ectodermal dysplasia-cleft lip/palate syndrome.

In some aspects, the compounds of formula (I), including each of the disclosed compounds and the compounds of formula (Ia), (Ib), (Ic), (Id), (Ie), and (If) may be administered through any route of administration, including but not limited to oral, nasal, intranasal, intramuscular, intravenous, subcutaneous, rectal, sublingual, intrathecal, transdermal, intraocularly, inhalation or other topical. In some aspects, the compounds of formula (I), including each of the disclosed compounds and the compounds of formula (Ia), (Ib), (Ic), (Id), (Ie), and (If), are administrated through intraocularly or topically to the eye. In some aspects, the pharmaceutically composition is an ophthalmic solution or suspension comprising the compound of formula (I) and one or more pharmaceutically acceptable excipients suitable for administration to the eye.

For any structure disclosed herein, the scope of a compound also includes any tautomer which may be formed. Unless otherwise indicated, reference to a compound should be construed broadly to include pharmaceutically acceptable salts, prodrugs, tautomers, alternate solid forms, non-covalent complexes, and combinations thereof, of a chemical entity of the depicted structure or chemical name.

A pharmaceutically acceptable salt is any salt of the parent compound that is suitable for administration to an animal or human. A pharmaceutically acceptable salt also refers to any salt which may form in vivo as a result of administration of an acid, another salt, or a prodrug which is converted into an acid or salt. A salt comprises one or more ionic forms of the compound, such as a conjugate acid or base, associated with one or more corresponding counter-ions. Salts can form from or incorporate one or more deprotonated acidic groups (e.g. carboxylic acids), one or more protonated basic groups (e.g. amines), or both (e.g. zwitterions).

A prodrug is a compound which is converted to a therapeutically active compound after administration. For example, conversion may occur by the removal of a biologically labile group. Prodrug preparation is well known in the art. For example, “Prodrugs and Drug Delivery Systems,” which is a chapter in Richard B. Silverman, Organic Chemistry of Drug Design and Drug Action, 2d Ed., Elsevier Academic Press: Amsterdam, 2004, pp. 496-557, provides further detail on the subject.

Tautomers are isomers that are in rapid equilibrium with one another. For example, tautomers may be related by transfer of a proton, hydrogen atom, or hydride ion.

Unless stereochemistry is explicitly depicted, a structure is intended to include every possible stereoisomer, both pure or in any possible mixture.

Alternate solid forms are different solid forms than those that may result from practicing the procedures described herein. For example, alternate solid forms may be polymorphs, different kinds of amorphous solid forms, glasses, and the like.

Non-covalent complexes are complexes that may form between the compound and one or more additional chemical species that do not involve a covalent bonding interaction between the compound and the additional chemical species. They may or may not have a specific ratio between the compound and the additional chemical species. Examples might include solvates, hydrates, charge transfer complexes, and the like.

When ranges of values are disclosed, and the notation “from n1 . . . to n2” or “n1 . . . to n2” is used, where n1 and n2 are the numbers, then unless otherwise specified, this notation is intended to include the numbers themselves and the range between them. This range may be integral or continuous between and including the end values. By way of example, the range “3 to 11 membered cycloalkyl” is intended to include cycloalkyl having three, four, five, six, seven, eight, nine, ten, or eleven ring atoms. When n is set at 0 in the context of “0 carbon atoms”, it is intended to indicate a bond or null.

As used herein, either alone or in combination, the term “alkyl” refers to a functional group comprising a straight-chain or branched-chain hydrocarbon containing from 1 to 20 carbon atoms linked exclusively by single bonds and not having any cyclic structure. An alkyl group may be optionally substituted as defined herein. Examples of alkyl groups includes, without limitation methyl, ethyl, n-propyl, isopropyl, n-butyl, isobutyl, sec-butyl, tert-butyl, pentyl, isoamyl, hexyl, heptyl, octyl, nonyl, decyl, undecyl, dodecyl, tridecyl, tetradecyl, pentadecyl, hexadecyl, heptadecyl, octadecyl, nonadecyl, eicosyl, and the like.

As used herein, either alone or in combination, the term “alkenyl” refers to a functional group comprising a straight-chain or branched-chain hydrocarbon containing from 2 to 20 carbon atoms and having one or more carbon-carbon double bonds and not having any cyclic structure. An alkenyl group may be optionally substituted as defined herein. Examples of alkenyl groups include, without limitation, ethenyl, propenyl, 2-methylpropenyl, butenyl, 1,4-butadienyl, pentenyl, hexenyl, heptenyl, octenyl, nonenyl, decenyl, undecenyl, dodecenyl, tridecenyl, tetradecenyl, pentadecenyl, hexadecenyl, heptadecenyl, octadecenyl, nonadecenyl, eicosenyl, and the like. The point of attachment can be on the double bond carbon or on any single bond carbon.

As used herein, either alone or in combination, the term “alkynyl” refers to a functional group comprising a straight-chain or branched-chain hydrocarbon containing from 2 to 20 carbon atoms and having one or more carbon-carbon triple bonds and not having any cyclic structure. An alkynyl group may be optionally substituted as defined herein. Examples of alkynyl groups include, without limitation, ethynyl, propynyl, hydroxypropynyl, butynyl, butyn-1-yl, butyn-2-yl, 3-methylbutyn-1-yl, pentynyl, pentyn-1-yl, hexynyl, hexyn-2-yl, heptynyl, octynyl, nonynyl, decynyl, undecynyl, dodecynyl, tridecynyl, tetradecynyl, pentadecynyl, hexadecynyl, heptadecynyl, octadecynyl, nonadecynyl, eicosynyl, and the like. The point of attachment can be on the triple bond carbon or on any single bond carbon.

As used herein, either alone or in combination, the term “alkoxy” refers to —O-alkyl, —O-alkenyl, or —O-alknyl, wherein alkyl, alkenyl, and alkynyl are as defined above.

As used herein, either alone or in combination, the term “alkoxyalkyl” means an alkyl as defined above substituted with an alkoxy group as defined above (in one aspect one or two alkoxy groups). C₂₋₆ alkoxyalkyl means the total number of carbon atoms. Examples include but not limited to 2-methoxyethyl, 1-, 2-, or 3-methoxypropyl, 2-ethoxyethyl, and the like.

As used herein, either alone or in combination, the term “aryl” refers to monocyclic, bicyclic (fused), and tricyclic (fused or spiro) hydrocarbon ring system having a total of five to fourteen ring atoms. When aryl is monocyclic, the monocyclic is aromatic and contains no heteroatom. When aryl is bicyclic or tricyclic, at least one of the ring in the bicyclic or tricyclic is aromatic and contains no heteroatom, and when the other ring(s) is aromatic, the other ring(s) does not contain a heteroatom, but when the other ring(s) is not aromatic, the other ring(s) may or may not contain a heteroatom. The point of attachment can be on any ring atom. Examples of aryl include, without limitation, benzene, naphthalene, indane, 1,2,3,4-tetrahydronaphthalene, chromane, isochromane, 1,2,3,4-tetrahydroquinoline, thiochromane 1,1-dioxide, 6,7,8,9-tetrahydro-5H-benzo[7]annulene, and 2,3-dihydrobenzofuran.

As used herein, either alone or in combination, the term “aralkyl” refers to a 5 to 12 membered heteroaryl or 6 to 12 membered aryl, as defined herein, substituted for a hydrogen of an C₁₋₆ alkyl.

As used herein, either alone or in combination, the term “cycloalkyl” refers to a monocyclic, bicyclic (fused, bridged, or spiro), or tricyclic (fused or spiro) hydrocarbon ring system having a total of three to fourteen ring atoms, which is completely saturated or contains one or more units of unsaturation, but none of the individual ring in the monocyclic, bicyclic, or tricyclic hydrocarbon is aromatic, and none of the ring atoms is a heteroatom. The point of attachment can be on the saturated or unsaturated carbon. A bridged bicyclic cycloalkyl refers to two hydrocarbon rings share three or more carbon atoms, separating the two bridgehead carbon atoms by a bridge containing at least one atom. Examples of cycloalkyl include, but not limited to, cyclopropane, cyclobutane, cyclopentane, cyclohexane, bicyclo[2.2.2]octane, bicyclo[2.2.1]heptane, spiro[2.5]octane, spiro[3.5]nonane, spiro[4.5]decane, and spiro[5.5]undecane.

As used herein, either alone or in combination, the term “haloalkyl” refers to alkyl as defined above, wherein one or one to five hydrogens are replaced with halogen atom(s), including those substituted with different halogen atoms. Examples of haloalkyl includes, but not limited to, —CF₃, —CH₂Cl, —CHF₂, and —CF₂CF₃.

As used herein, either alone or in combination, the term “haloalkoxy” refers to alkoxy as defined above, wherein one or one to five hydrogens are replaced with halogen atom(s), including those substituted with different halogen atoms. Examples of haloalkoxy includes, but not limited to, —OCF₃ and —OCHF₂.

As used herein, alone or in combination, the term “halo” or “halogen” means fluoro, chloro, bromo, or iodo; in one aspect, fluoro or chloro.

As used herein, either alone or in combination, the term “spiro” refers to a moiety comprising two rings sharing one common atom.

### DESCRIPTION OF FIGURES

FIG. 1A shows various concentrations of CAP1160 exposed to UV.

FIG. 1B shows absorbance at 600 nm for various concentrations of CAP1160 exposed to UV.

### EXAMPLES

### Example 1: Prevention of UVC/H₂O₂-Induced Aggregation of Bovine Lens Extracts by CAP1160

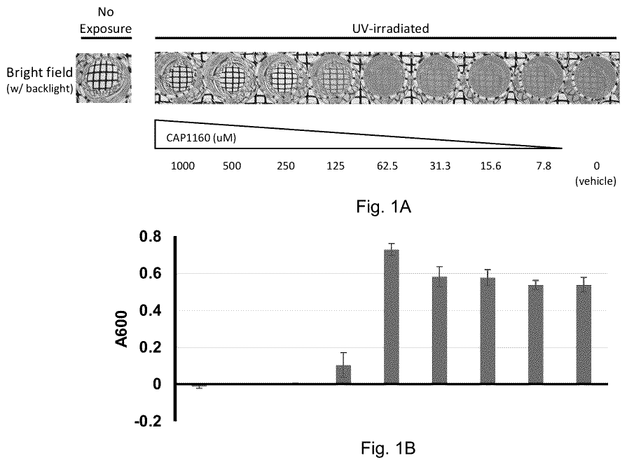

Bovine lens lysates (2 mg/ml, 50 μl) were incubated with various concentrations of CAP1160 (structure shown below) or a vehicle (0.5% DMSO), and then left unexposed (no exposure) or exposed to UV (UV-irradiated). See FIG. 1A. At various time points, wells-containing bovine lens lysates were pictured and the corresponding absorbance at 600 nm (A600) was measured. See FIGS. 1A and 1B. Representative brightfield images of wells taken 7 min after initial UV exposure (FIG. 1A) and corresponding A600 (FIG. 1B).

The results show that CAP1160 delays, in a dose-dependent manner, the opacification of bovine lens protein lysates induced by UV irradiation.

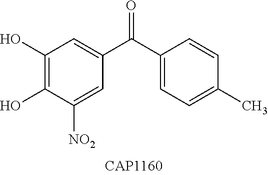 [SMILES: Cc1ccc(C(=O)c2cc(O)c(O)c([N+](=O)[O-])c2)cc1]

### Example 2: General Scheme for Making Prodrugs

Prodrugs described herein are prepared by the following general scheme.

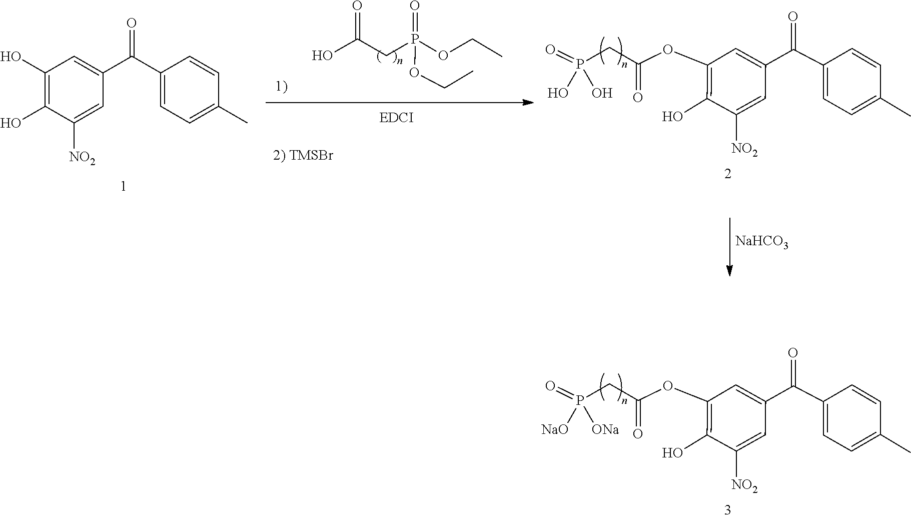

In the above scheme, starting compound 1 and the reagent used for reacting with the OH group are mere examples, and each may be selected as appropriate based on the desired end product.

1. A compound, where in the compound is: 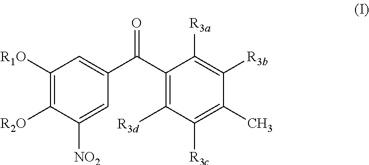
or a solvate or a pharmaceutically acceptable salt thereof,
wherein,
R₁ and R₂ are the same or different, and each of R₁ and R₂ is independently selected from the group consisting of: hydrogen, R₄C═O, 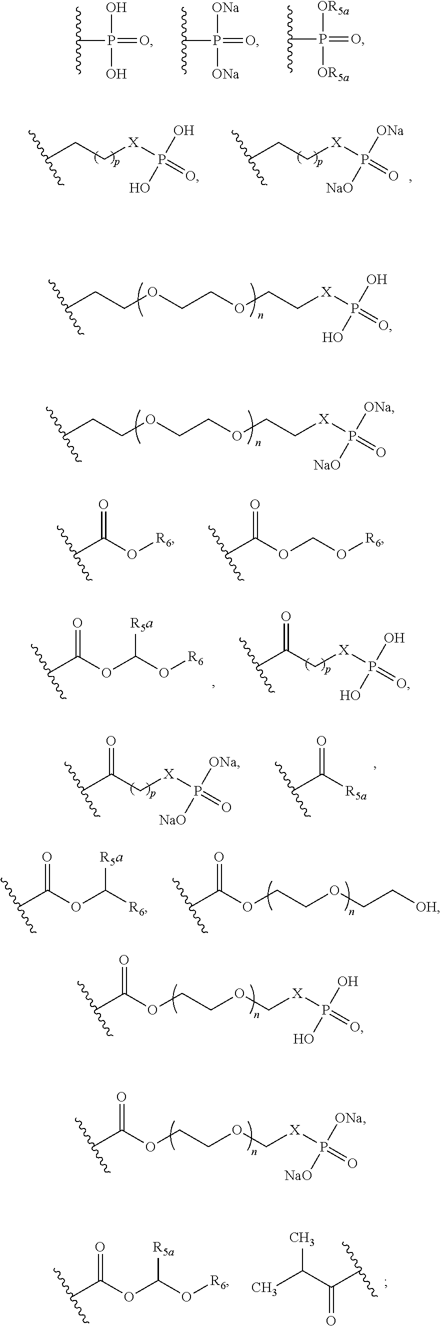
R₃ₐ, R_{3b}, R_{3c}, and R_{3d} are each the same or different, and each independently are selected from the group consisting of: hydrogen, branched or linear (C₁-C₆)alkyl, halo(C₁-C₆)alkyl, (C₃-C₆)cycloalkyl, halo(C₃-C₆)cycloalkyl, and hydroxyl;
R₄ is selected from the group consisting of: branched or linear (C₁-C₆)alkyl; halo(C₁-C₆)alkyl; (C₃-C₆)cycloalkyl; halo(C₃-C₆)cycloalkyl; aryl; haloaryl; and
R₅ₐ and R_{5b} are each the same or different, and independently a branched or linear (C₁-C₆)alkyl,
R₆ is branched or linear (C₁-C₆)alkyl, aryl, or a polyethylene glycol group or 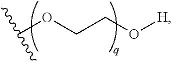
wherein q is 1 to 10;
p is a number from 0 to 10,
n is a number from 0 to 10, and
X is a C or O;

2. The compound of claim 1, wherein at least one of R₁ and R₂ is selected from the group consisting of 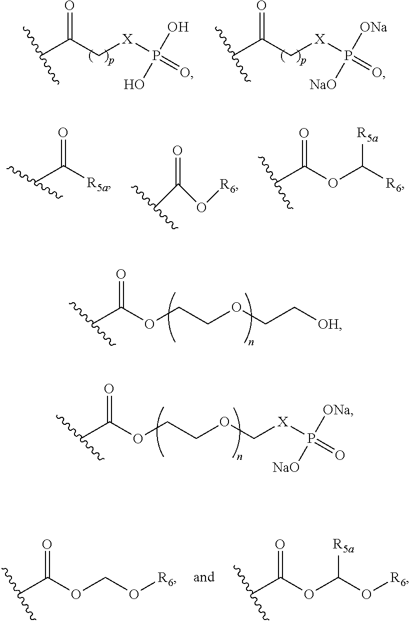
wherein R₅ₐ is (C₁-C₆)alkyl and R₆ is (C₁-C₆)alkyl, aryl, or, 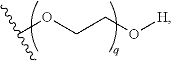
wherein q is 1 to 10,
or a solvate or a pharmaceutically acceptable salt thereof.

3. A compound of formula (Ia): 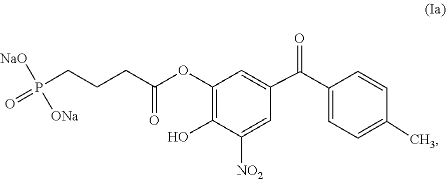
or a solvate or a pharmaceutically acceptable salt thereof;
a compound of formula (Ib): 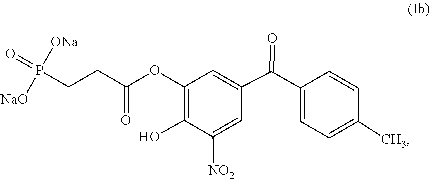
or a solvate or a pharmaceutically acceptable salt thereof;
a compound of formula (Ic): 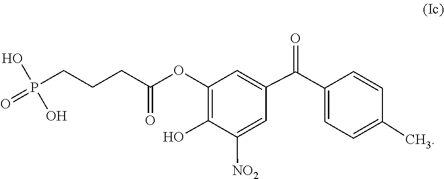 [SMILES: Cc1ccc(C(=O)c2cc(OC(=O)CCCP(=O)(O)O)c(O)c([N+](=O)[O-])c2)cc1]
or a solvate or a pharmaceutically acceptable salt thereof;
a compound of formula (Id); 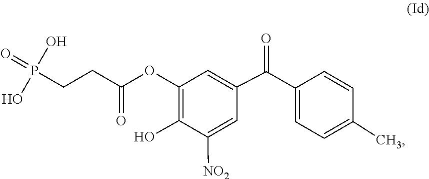 [SMILES: Cc1ccc(C(=O)c2cc(OC(=O)CCP(=O)(O)O)c(O)c([N+](=O)[O-])c2)cc1]
or a solvate or a pharmaceutically acceptable salt thereof;
a compound of formula (Ie): 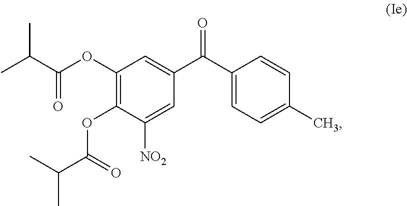 [SMILES: Cc1ccc(C(=O)c2cc(OC(=O)C(C)C)c(OC(=O)C(C)C)c([N+](=O)[O-])c2)cc1]
or a solvate or a pharmaceutically acceptable salt thereof; or
a compound of formula (If): 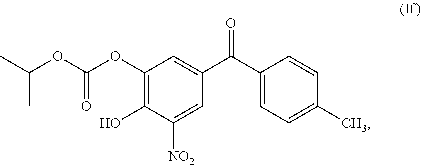 [SMILES: Cc1ccc(C(=O)c2cc(OC(=O)OC(C)C)c(O)c([N+](=O)[O-])c2)cc1]
or a solvate or a pharmaceutically acceptable salt thereof.

4. (canceled)

5. (canceled)

6. (canceled)

7. (canceled)

8. (canceled)

9. A compound selected from: 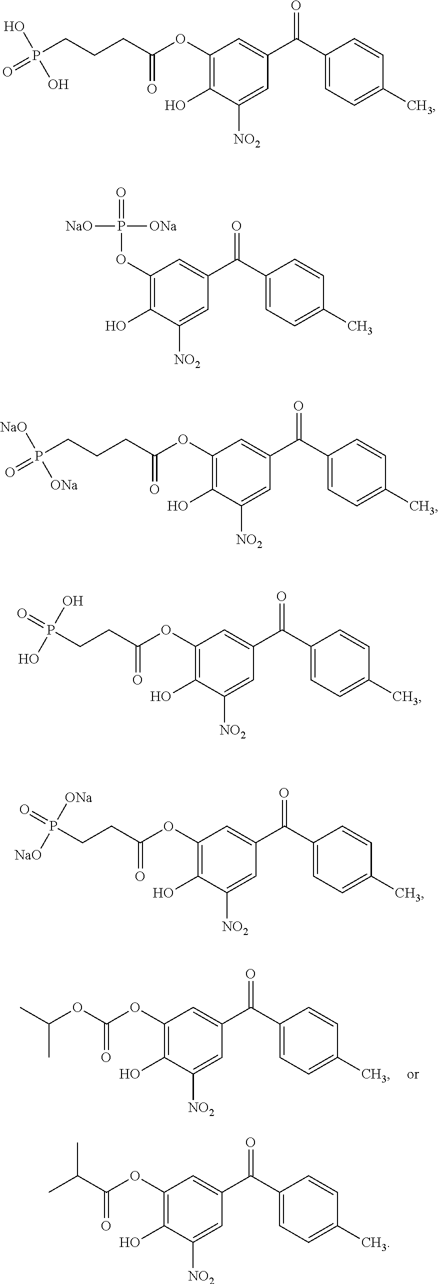
or a solvate or a pharmaceutically acceptable salt thereof.

10. A pharmaceutical composition comprising a compound of claim 1 and one or more pharmaceutically acceptable excipients.

11. A method of treating, preventing, reducing the occurrence or, or reducing, ameliorating, or alleviating the symptoms associated with presbyopia, cataract, transthyretin (TTR)-associated amyloidosis, or other conditions or disorders associated with the eye, comprising administering to a subject in need thereof a compound of claim 1.

12. A method of treating, preventing, reducing the occurrence or, or reducing, ameliorating, or alleviating the symptoms associated with presbyopia, cataract, transthyretin (TTR)-associated amyloidosis, or other conditions or disorders associated with the eye, comprising administering to a subject in need thereof the pharmaceutical composition of claim 10.
